(19) United States
(12) Patent Application Publication
(10) Pub. No.: US 2022/0000568 A1
(43) Pub. Date: Jan. 6, 2022

(54) System and Method for Modulating Tissue Retraction Force in a Surgical Robotic System

(71) Applicant: TransEnterix Surgical, Inc., Morrisville, NC (US)

(72) Inventors: Kevin Andrew Hufford, Cary, NC (US); Matthew Robert Penny, Holly Springs, NC (US)

(21) Appl. No.: 16/920,737

(22) Filed: Jul. 5, 2020

(51) Int. Cl. A61B 34/30; A61B 17/02; A61B 34/32; A61B 17/32; A61B 17/068; A61B 17/12; A61B 17/04

(52) U.S. Cl. CPC A61B 34/30; A61B 17/0206; A61B 34/32; A61B 2090/066; A61B 17/068; A61B 17/12; A61B 17/0469; A61B 17/32

(57) ABSTRACT

A surgical system and method for maintaining optimal surgeon-controlled tissue force or torque with a surgical robot in which a surgical instrument held by an additional robotic arm that is not being actively telemanipulated by a surgeon applies a force or torque to tissue.

This application claims the benefit of U.S. Provisional Application No. 62/503,062, filed May 9, 2017.

### BACKGROUND

There are various types of surgical robotic systems on the market or under development. Some surgical robotic systems use a plurality of robotic arms. Each arm carries a surgical instrument, or the camera used to capture images from within the body for display on a monitor. See U.S. Pat. No. 9,358,682. Other surgical robotic systems use a single arm that carries a plurality of instruments and a camera that extend into the body via a single incision. See WO 2016/057989. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a master console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

US Patent Publication US 2010/0094312 (the '312 application) describes a surgical robotic system in which sensors are used to determine the forces that are being applied to the patient by the robotic surgical tools during use. This application describes the use of a 6 DOF force/torque sensor attached to a surgical robotic manipulator as a method for determining the haptic information needed to provide force feedback to the surgeon at the user interface. It describes a method of force estimation and a minimally invasive medical system, in particular a laparoscopic system, adapted to perform this method. As described, a robotic manipulator has an effector unit equipped with a six degrees-of-freedom (6-DOF or 6-axes) force/torque sensor. The effector unit is configured for holding a minimally invasive instrument mounted thereto. In normal use, a first end of the instrument is mounted to the effector unit of the robotic arm and the opposite, second end of the instrument (e.g. the instrument tip) is located beyond an external fulcrum (pivot point kinematic constraint) that limits the instrument in motion. In general, the fulcrum is located within an access port (e.g. the trocar) installed at an incision in the body of a patient, e.g. in the abdominal wall. A position of the instrument relative to the fulcrum is determined. This step includes continuously updating the insertion depth of the instrument or the distance between the (reference frame of the) sensor and the fulcrum. Using the 6 DOF force/torque sensor, a force and a torque exerted onto the effector unit by the first end of the instrument are measured. Using the principle of superposition, an estimate of a force exerted onto the second end of the instrument based on the determined position is calculated. The forces are communicated to the surgeon in the form of tactile haptic feedback at the hand controllers of the surgeon console.

During the course of surgical procedures, it is necessary for the surgeon to place tissues under tension so that s/he can then dissect, cut, or perform some other step involving that tissue. During dissection or other such surgical procedures, it is often advantageous to use three instruments, one for traction, one for dissection (scissors/ultrasonic/electrosurgical, etc.), and one for counter-traction. The tension resulting from the two opposing traction forces on the tissue being dissected improves the cut quality and speed of the dissection.

For example, referring to FIG. 1, to dissect the tissue around the fundus of the stomach during a Nissen fundoplication, the surgeon will use a first instrument 1 to apply traction T1 to the tissue of the stomach and a second instrument 2 to apply traction T2 to the fascia at the fundus. The tension resulting from the traction forces T1, T2 makes the target tissue sufficiently taut to allow dissection using a dissecting instrument 3 as shown. As noted, the use of two opposing forces on the tissue being dissected improves the cut quality and speed of the dissection

Referring to FIG. 4, with some typical surgical robotic systems, a surgeon console 12 has two input devices such as handles 17, 18 that the surgeon selectively assigns to robotic arms 14, 16, 17, allowing surgeon control of two of the surgical instruments 10a, 10b, and 10c disposed at the working site at any given time. To control a third instrument disposed at the working site, one of the two handles 17, 18 is operatively disengaged from one of the initial two instruments and then operatively paired with the third instrument. (Note that in FIG. 4 the laparoscopic camera, which may be a robotically positioned camera supported by a fourth robotic arm, is not shown for purposes of clarity.) When performing a procedure of the type described above using robotically driven instruments, the surgeon might use the input handles 17, 18 to move the two instruments 1, 2 used for traction T1 and T2 into position engaging the tissue with desired force vectors for the traction. Once the traction forces are applied by the instruments, the surgeon re-assigns one of the input handles 17, 18 so that it is paired with the dissection instrument 3 (e.g. scissors or other forms of cutting or separating instruments, including those employing an energy modality). The instrument (for example, instrument 1) that is operatively disengaged from an input handle remains in a fixed position and orientation. The surgeon may then use one of the input handles 17, 18 to control the dissection instrument 3, and use the other one of the input handle 17, 18 to adjust the position and orientation of the retractor 2 that remains operatively associated with an input handle. Using this technique, the advancement of the dissection instrument must be periodically interrupted to switch control of a handle to the arm supporting instrument 1 in order to move instrument 1 sufficiently to adjust the tension, and then re-associate that handle with the dissection instrument 3 to allow surgeon control of the dissecting instrument. Alternatively, the surgeon might control one of the instruments 1 from the console for purposes of applying traction, and no second robotically positioned retractor is used. Instead, a surgical assistant manually controls a manual second retractor to provide the counter-traction. Results using this method may vary based on the assistant's skills and the communication from the surgeon.

This application describes aspects of a surgical robotic system that allow tissue retraction forces to be automatically modulated where, as above, the surgeon is unable to actively control forces applied by both the retraction instruments.

### BRIEF DESCRIPTION OF THE DRAWINGS

FIG. 1 schematically illustrates use of traction and counter-traction in a laparoscopic surgical procedure.

FIGS. 2 and 3 schematically illustrate the use of robotically-controlled traction and surgeon-controlled traction according to an aspect of the disclosed invention.

FIG. 4 schematically illustrates elements of a surgical robotic system of a type that may be adapted for use with the disclosed invention.

FIGS. 5A through 6 illustrated mode of operation of the described systems.

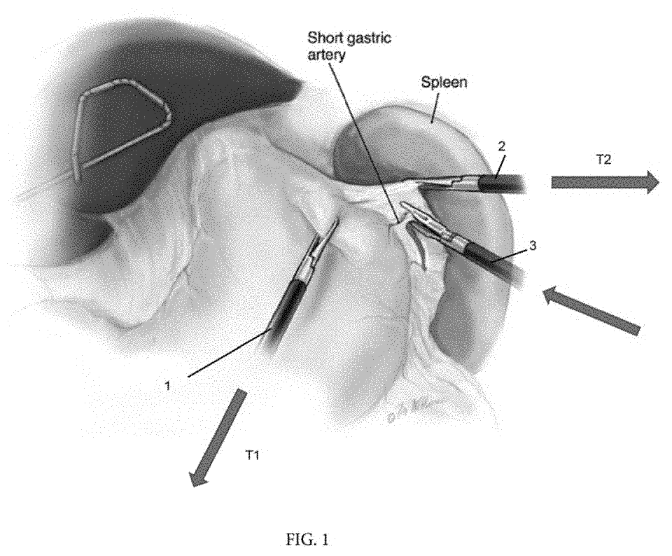

### DETAILED DESCRIPTION

The present application describes a system and method for maintaining optimal tissue tension between instruments of a surgical robotic system.

The surgical system may be of a type described in the Background, or any other type of robotic system used to maneuver surgical instruments at an operative site within the body. The surgical system is one configured to determine or estimate the forces and/or torque each robotically manipulated surgical instrument applies to tissue. It thus may include one or more sensors positioned to estimate or determine the. The system might employ one or more single-axis sensors, multi-axis sensors, or a combination of single- and multi-axis sensors. Exemplary positions for the sensor(s) include the instrument tip, instrument shaft, the robotic arm or some combination of these positions. Types of sensor arrangements that can be used include, without limitation, those described in US Patent Publication US 2010/0094312 or PCT/US2017/015691, as well as other configurations. Other embodiments might derive force information by reading the motor currents at the joints of the robotic manipulator arm, a configuration that also will be referred to as a “sensor” for the purposes of this description. It should be understood that the scope of the invention is not limited to any particular type of sensor type or location, as the invention may be practiced using any sensor type or sensor location that will allow the system to determine the force or torque apply by a surgical instrument to tissue.

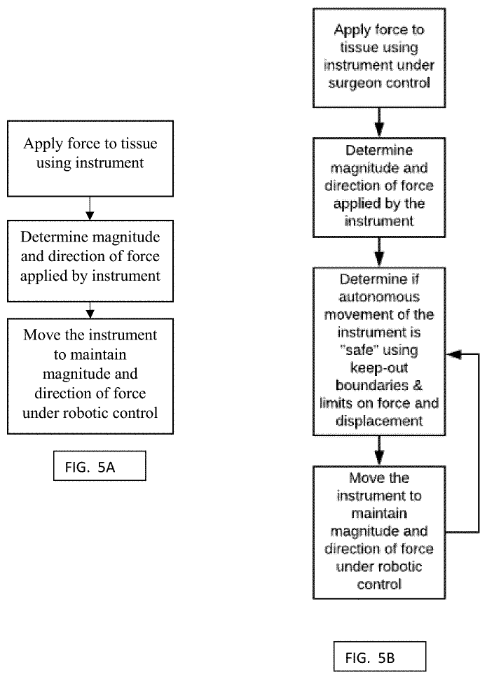

A simple embodiment incorporating principles of the present invention is depicted in the flow chart of FIG. 5A. This embodiment is one in which force or torque applied by an instrument to tissue is autonomously modulated in a direction and magnitude that is set by the surgeon. Using this embodiment, a first instrument is caused to apply a force to the tissue. The first instrument may be one that grasps tissue and the force may be applied to place the tissue under tension or to apply torque to the tissue. Alternatively, the first instrument may be a retractor of a type having a surface (e.g. a fan retractor or alternative configuration) used to press or push tissue away from an area of interest. In either case, the system is preferably configured so that the user moves the first instrument to the tissue and causes the first instrument to apply the force/torque to the tissue.

This initial step may be performed by a user who manipulates a control handle at the surgeon console to command the robotic arm to navigate the first instrument to the tissue and to apply the force having the desired magnitude and direction to the tissue (“surgeon control.”). An instruction is given to the system to “set” this force magnitude and direction for use in force maintenance mode, described below.

This initial step need not be performed under surgeon control. Instead, a user standing adjacent to the patient may manually guide the robotic arm and first instrument into a desired position, and to move the instrument into contact with the tissue to apply the force/torque. Depending on the instrument and the capabilities of the robotic system, this might involve using a manual actuator to close the jaws of a grasping instrument onto tissue and then moving the arm to apply the force, or by manually moving the arm to push a retractor against the tissue. The direction of the force might then be set by aligning the shaft of the instrument along the desired axis of force (e.g. if traction is to be applied by the instrument), or an input device can be used to instruct the system as to the desired direction of force with respect to the endoscopic image of the instrument on the endoscopic display. For example, an eye tracking device positioned to receive input based on movement of the user's eyes across the endoscopic image display might be activated to record eye motion as the user traces the intended direction of force with his/her eyes along the display. Alternatively, the user might move a cursor across the display or employ a touch input or/overlaying the display to set the direction.

Next, the user places the system in a force maintenance mode, under which the magnitude and direction of the force or torque set by the user or imparted on the tissue by the first instrument is dynamically maintained under robotic control. The force is maintained through autonomous movement of the first instrument using the robotic arm. While the system is in force maintenance mode, the user causes other surgical instruments (manual instruments or those manipulated using robotic arms) to act on the tissue in ways that might cause variation in the magnitude of the force/torque applied by the first instrument had it not been placed in force maintenance mode.

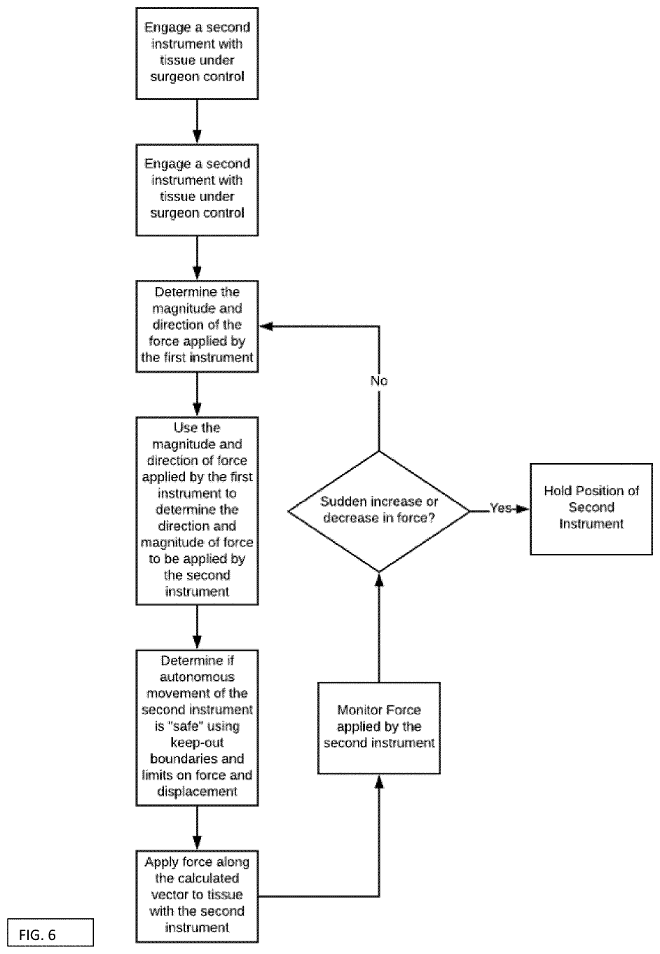

This embodiment might be modified so that a sudden decrease in the magnitude of the force (i.e. a decrease in the force by more than a pre-determined amount within a predetermined period of time) might cause the system to hold the position of the first instrument rather than attempting to maintain the force. This feature is discussed in further detail in connection with the embodiment of FIG. 6.

FIG. 5B shows a modification to FIG. 5A in which limits are placed on the autonomous movement of the first instrument. In this example, before the robotic manipulator moves the first instrument to maintain the force, it first determines whether it is safe to do so. This step may take various forms. As one example, the system might be programmed (or instructed by the user using the user interface) to prevent movement of the instrument more than a predetermined maximum displacement in any direction or in particular directions. As another example, the system might include features that allow the user to define “keep out” boundaries with reference to the endoscopic image display and instruct the system to prevent movement of the instrument tip beyond those boundaries.

The system can be configured to receive input defining the boundaries in a number of different ways. For example, the user might trace the boundaries using a touch interface on or over the endoscopic image display, or using input from an eye tracker generated during movement of the user's eyes over the endoscopic display to define the boundaries. In these examples, the depth of the bound space might be defined using a variety of means, such as by placing the tip of an instrument such as the endoscopic camera at the maximum depth to be input to the system. In other examples, the keep-out boundaries might be defined by the user prior to the procedure using models generated using pre-operative images or scans, or the system might be configured to recognize delicate tissue structures using computer vision and to register the areas containing such structures as keep-out zones. Another pre-set or user settable limit might include maximum force limits.

If the movement needed to maintain the magnitude of the force is determined to be safe, the robotic arm autonomously moves the instrument. If the system determines that the movement would not be safe, the system alerts the user (e.g. using auditory, visual or tactile feedback) and does not move the instrument beyond the safe limits.

In a multi-arm robotic system making use of the configuration described with reference to FIG. 5A or FIG. 5B, the system may be operable with one arm in force maintenance mode, or with more than one arm in force maintenance mode. Where two or more arms operable in force maintenance mode, the user may operate the system such that at least two arms are operating in force maintenance mode at one time, so that each arm is independently functioning to maintain the force/torque and direction set by the user for the associated instrument.

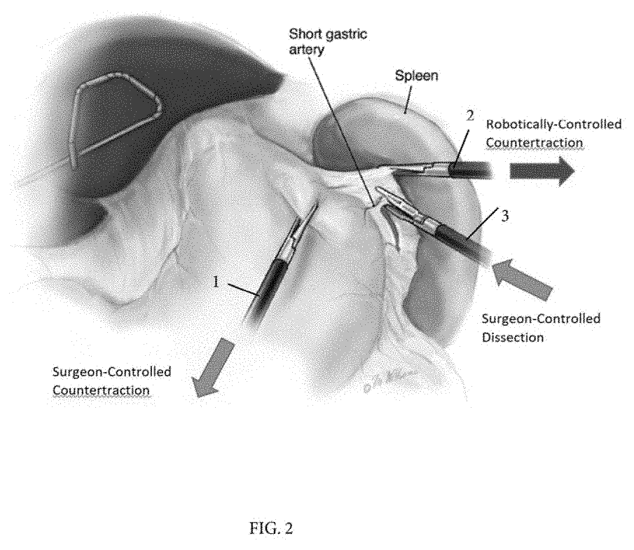

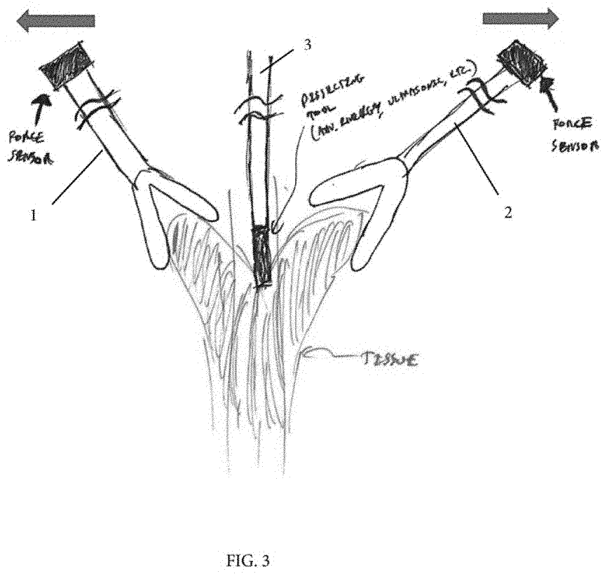

A second embodiment will next be described with reference FIGS. 2 and 6. The second embodiment is one in which force or torque applied by an instrument to tissue is autonomously modulated in a direction and magnitude that is determined by the force magnitude and direction applied by a different instrument (one that is preferably under surgeon control). This discussion will describe the second embodiment in the context of application of traction and countertraction using grasping retractors to maintain tissue tension during tissue dissection. It should be understood, however, that it may be used with other types of instruments, it may be used in connection with forces other than traction and countertraction forces, and it may be used where the action to be taken on the tissue is an action other than dissection.

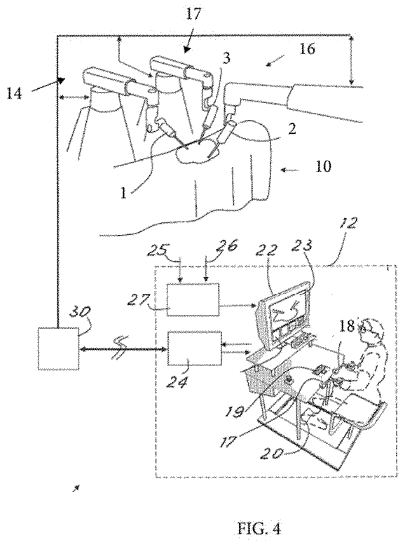

In this embodiment, first and second instruments 1, 2 (such as retractors) are positioned in a surgical workspace and used to engage or contact to tissue. The steps of positioning the instruments in the workspace and engaging the tissue are preferably performed by the surgical robotic system based on active surgeon input (“surgeon control”, although manual positioning might instead be used as discussed above. For example, the surgeon might use a first input device (e.g. a first input handle 17, FIG. 4) to cause the system to position the first traction instrument 1 and engage tissue with that instrument, and a second input device (e.g. a second input handle 18, FIG. 4) to cause the system to position the second traction instrument 2 and engage tissue with the second traction instrument. As a second alternative, the first input device might be used to position/engage the first traction instrument, and separately used to do the same for the second traction instrument. Using this second alternative, after one traction instrument is initially positioned using input from the input device, the input device is then paired with and used to position the other traction instrument. Manual positioning of the instruments using steps of the type described in connection with the first embodiment might instead be used for one or both of the instruments.

After the instruments are positioned and used to contact or engage tissue, the system is operated in a force or torque modulation mode (which in this embodiment is a traction modulation mode). In this mode, the system is operated so that one instrument may be actively telemanipulated by the surgeon using an input device (which will be referred to here as “surgeon-controlled” to mean that the robotic surgical system controls motion of the traction instrument to move it based on input from the surgeon). The other instrument is not actively telemanipulated by a surgeon but applies a force or torque to tissue under control of the robotic system. This type of control will be referred to here as “autonomously controlled” to mean that the robotic surgical system is not directly responding to surgeon input from an input device paired with the traction instrument to control the position of the second instrument. It instead automatically controls force/torque (in this specific example countertraction forces) applied by the second instrument based on other parameters. In preferred embodiments, the direction and magnitude of the applied force of the autonomously-controlled instrument is determined by the robotic system based on the direction and magnitude of the force of the surgeon-controlled instrument as it is actively telemanipulated by the surgeon.

As countertraction is applied to tissue by the first and second traction instruments 1, 2, a third, preferably surgeon-controlled, instrument 3 is used to perform a procedure on the tissue. The third instrument may be a treatment instrument such as a dissection instrument that separates tissue using blades, energy, forces, or some other modality, or it might be some other type of instrument such as a stapler, ligation instrument, suturing instrument, sealing instrument, etc.

One embodiment of the described system might make use of components of a system of the type shown in FIG. 4. In this embodiment, the first traction instrument 1 is mounted to a first robotic arm 14 that is being actively telemanipulated by a surgeon operating a first input device 17. The surgeon manipulates the input device 17 to control movement of the arm 14 and thus the countertraction forces applied to tissue by the first traction instrument. The second traction instrument 2 is mounted to a second robotic arm that, when the system is in the modulation mode, is robotically-controlled to cause the second traction instrument to provide countertraction to the tissue. The system determines the magnitude and direction of the force or torque applied to tissue by the instrument on the first arm 14. This may be carried out using one or more force sensors in or on the first arm 14 or the first instrument as described above. In some configurations sensors may be used to determine the magnitude while other system features, such as the system kinematics, are used to determine the direction.

Using the measured or derived direction and magnitude of the force/torque applied to tissue by the first instrument, the system determines a complementary force/torque magnitude and direction to be applied to tissue by the second instrument. As an example of this embodiment, when this mode of operation is triggered, the second arm 16 will cause the second traction instrument 2 to pull the tissue using a force that is of substantially equal magnitude and that has a direction that is mirrored relative to the direction of movement of the first instrument relative to a defined plane. The plane may be defined relative to the camera vector or a tissue plane, or it may be at a location defined by the user using input techniques described elsewhere in this application. In some cases the force/torque applied to the second instrument might be of substantially equal force and/or in an equal and opposite direction from the first traction instrument 1 on the first arm. Determining the complementary magnitude and direction might take into account other parameters such as the type or properties of the tissue receiving force/torque from the first or second instruments and other factors.

The autonomous modulation may be limited by a not-to-exceed force value as described with respect to the first embodiment. The not-to-exceed force value may be statically set, be dynamically determined, or selectively set by the user.

The travel of the robotically-controlled traction instrument may also be limited by certain travel limits. This may take the form of a maximum-allowable deviation from the instrument's current position, the avoidance of static or dynamically-set “keep-out” zones (into which the instrument will not be permitted to pass), or any combination thereof. See the description of the first embodiment.

In one implementation, the second arm causes the second instrument 2 to apply the same force to the tissue that the first instrument held by the surgeon-controlled first arm applies. In another implementation, the instrument held by the second arm maintains a set tension, with a not-to-exceed limit. This value may be set once prior to the procedure, may be adjusted by the surgeon during the procedure, or may be dynamically set during the procedure and calculated by the surgical robot.

With traction and counter-traction applied, the surgeon carries out the dissection procedure using instrument 3, which is manipulated by arm 17, in accordance with user input using input device 18, while the system in traction modulation mode provides continuous, optimal tension adjustments through the robotically-controlled movement of instrument 2.

In preferred implementations, a hybrid of force control and position control is used to prevent overshoot of the robotically-controlled traction instrument 2 upon a sudden decrease of tension resulting from a release of tissue or the cutting of a specimen, such as during dissection or cutting using instrument 3. In the embodiment depicted in FIG. 6, when the system detects a sudden decrease in traction force applied by the first instrument, the system holds the position of the second instrument.

In other implementations, the system is configured so that the forces/torque (in this embodiment traction and counter-traction) applied by the instruments 1, 2 may be automatically applied by robotically-controlled arms modulated to a safe value.

Another application for the principles described herein is one in which a robotically-controlled arm may be used to provide tissue tension during suturing, provide optimal apposition of tissue at the suture line, etc. In this example, a first instrument is surgeon controlled to manipulate a first tissue, and a second instrument is autonomously manipulated using force/torque having a magnitude and direction determined based on the magnitude and direction of the force/torque applied by the first instrument, with the effect of maintaining the first and second tissues in apposition during tissue suturing or other forms of fastening or attachment.

In another embodiment, autonomously modulated force/torque is used in some procedure-specific contexts. For example, the system might be programmed to move an instrument that is applying force to tissue along a path or according to a sequence that is based on the surgical procedure being performed. For example, in a cholecystectomy procedure, a first instrument is used to grasp and elevate the gallbladder. A second instrument is used to dissect the gallbladder from the liver bed. As the dissection progresses, it can be beneficial to change the magnitude and direction of the load applied to the gallbladder according to a sequence that is pre-defined or dynamically defined based on keep-out zones and other parameters. A sequence of this type might include changing the nature of the force from a lifting force to a rotational force during the course of the sequence (e.g. as the gallbladder is being rotated off the liver bed).

Although the above embodiments describe surgical instruments on separate robotic arms, systems such as those in WO 2016/057989, which use of a plurality of surgical devices disposed on a single arm, can also be used with the present invention. For example, the first and second traction instruments may be retractors that bend or articulate using robotically driven actuators. In use, a first, surgeon-controlled, traction force is applied to tissue by bending/articulation of one of the retractors engaged with tissue, based on surgeon input using a user input device. A second, robotically-controlled, retraction force is applied to the tissue by the other one of the retractors which is caused to bend/articulate so as to apply the second force having direction and magnitude determined by the system using principles described above. A third, surgeon-controlled, instrument is used to perform the dissection.

The disclosed inventions provide several advantages over systems and methods of the prior art, including automation of the maintenance of force on tissue (of tension on tissue while dissecting), use of the force sensors in the robotic arms to aid in the maintenance of tension between two instruments, and the combined use of force control and position control to maintain safe positions of the robot, especially when tissue tearing, dissection, or detachment from tissue occurs.

1. A medical robotic system comprising:
at least a first robotic arm configured to support a surgical instrument having an end effector,
a sensor positioned to determine a magnitude of force or torque applied to tissue of a patient using the instrument;
wherein the robotic system includes a mode of operation operable to dynamically adjust the end effector position within the patient using the first robotic arm to substantially maintain a target direction and magnitude of forces or torques applied by the end effector.

2. The system of claim 1, further including at least one input device operable to set the target direction and magnitude.

3. The system of claim 2, wherein the input device includes a user control input operable to command robotic motion of the robotic arm to position the end effector in contact with tissue and to apply a force or torque to the tissue at a first magnitude and in a first direction, and to instruct the system to set the target direction and target magnitude at the actual magnitude and actual direction.

4. The system of claim 2, wherein the first robotic arm includes a manual mode in which the end effector is manually movable by the hand of a user to position the end effector in contact with tissue and to apply an actual force or torque to the tissue at an actual magnitude, and wherein the system includes a surgeon input operable to instruct the system to set the target magnitude at the actual magnitude.

5. The system of claim 1, where the system includes:
a second robotic arm supporting a second instrument having a second end effector,
a user input operable to command robotic motion of the second robotic arm to position the second end effector in contact with tissue and to apply a force or torque to the tissue at a second magnitude and in a second direction
wherein the second robotic arm includes a mode of operation in which the second arm is controlled by a user using user input and the first arm is autonomously controlled to cause the second effector to apply force or torque to tissue using force or torque having a first direction and a first magnitude, the first direction and first magnitude determined by the system using the second direction and second magnitude.

6. The system of claim 5, wherein the in the mode of operation movement of the second effector mirrors movement of the first end effector relative to a defined plane.

7. The system of claim 1, where the system includes a plurality of arms, wherein at least two or more arms are enabled with the modulation mode, each arm being used to retract tissue, either along the sample plane (shared direction) or individually (different loads and directions).

8. The system of claim 1, where the movement allowed by the robotically controlled arm and the applied load is bound by operational limits that are controlled by the surgeon.

9. A surgical robotic system, comprising:
first and second robotic arms configured for robotic positioning of first and second surgical instruments, respectively, in a body cavity, the robotic system configured to measure or receive as input direction and value of forces and/or torques applied by an end effector of each surgical instrument in contact with tissue using input from at least one sensor on the corresponding arm;
at least one surgeon input device for receiving surgeon input, the robotic system configured to control the first robotic arm based on the surgeon input to apply a first force and/or torque to tissue using the first surgical instrument;
wherein the system is operable in a force modulation mode in which the robotic system controls the second robotic arm to apply a second force to the tissue using the second surgical instruments, the system configured to determine the direction and magnitude of the second force and/or torque to be applied based on the direction and magnitude of the first force and/or torque.

10. The system of claim 9, further including a third robotic arm configured for robotic positioning of a third surgical instrument based on surgeon input using the at least one surgeon input device.

11. The system of claim 10, wherein system includes first and second surgeon input devices, the robotic system configured to control the first robotic arm based on surgeon input using the first surgeon input device, and to control the third robotic arm based on surgeon input using the second surgeon input device.

12. The system of claim 11 wherein the first and second surgical instruments are traction instruments and the third surgical instrument is an instrument for cutting, separating, or dissecting tissue.

13. A surgical method comprising:
positioning an end effector of a first surgical instrument carried by a first robotic arm in engagement with tissue in a body cavity;
applying a force having a direction and magnitude to the tissue using the end effector; and
controlling movement of the first robotic arm using the robotic surgical system, to maintain the direction and magnitude of the force while the tissue is being treated using a second instrument carried by a second robotic arm.

14. The method of claim 13, wherein the first surgical instrument is a retractor and the second surgical instrument is an instrument for dissecting, cutting, sealing, ligating, stapling, or separating tissue.

15. The method of claim 13, wherein the first surgical instrument is a retractor and the second surgical instrument is a suture device.

16. The method of claim 13, wherein the method further includes:
positioning a second end effector of a second surgical instrument carried by a second robotic arm in engagement with tissue in a body cavity;
applying tension to the tissue by applying a first force to the tissue using the first robotic arm in response to user input using a surgeon input device, and applying a second force to the tissue using the second robotic arm, the direction and magnitude of the second force determined by the robotic system based on the direction and magnitude of the first force.
